(19) United States
(12) Patent Application Publication
(10) Pub. No.: US 2025/0213251 A1
(43) Pub. Date: Jul. 3, 2025

(54) CATHETERS, SYSTEMS AND METHODS FOR FORMING A FISTULA

(71) Applicant: TVA MEDICAL GROUP, INC., Franklin Lakes, NJ (US)

(72) Inventors: Oladipo Peter Akerle-ale, Franklin Lakes, NJ (US); Andrew Moll, Franklin Lakes, NJ (US); Alexander Palmer, Franklin Lakes, NJ (US); Olivia R. Palmer, Franklin Lakes, NJ (US); Kristin N. Rominger, Franklin Lakes, NJ (US); Breanna Simpson, Franklin Lakes, NJ (US)

(21) Appl. No.: 18/853,375

(22) PCT Filed: Apr. 1, 2022
(86) PCT No.: PCT/US2022/023020

§ 371 Date: Oct. 1, 2024

(51) Int. Cl. A61B 17/11; A61B 17/00; A61B 18/00

(52) U.S. Cl. CPC A61B 17/11; A61B 2017/00314; A61B 2017/00876; A61B 2017/1107; A61B 2017/1139; A61B 2018/00404; A61B 2018/00577

(57) ABSTRACT

A catheter for use in a system for forming a fistula, the catheter including a catheter body, and a segmented backstop coupled to the catheter body and configured to receive a fistula-forming element, wherein the segmented backstop comprises at least a proximal segment and a distal segment, independently articulable relative to one another.

### TECHNICAL FIELD

The present disclosure relates to catheters, systems, and methods for endovascular treatments of a blood vessel, and more particularly catheters, systems, and methods having segmented backstops.

### BACKGROUND

Endovascular treatments of a blood vessel may include fistula formation. A fistula is generally a passageway formed between two internal organs (e.g., blood vessels or other bodily organs). Forming a fistula between two blood vessels can have one or more beneficial functions. For example, the formation of a fistula between an artery and a vein may provide access to the vasculature for hemodialysis patients or other subjects. Specifically, forming a fistula between an artery and a vein allows blood to flow quickly between the vessels while bypassing the capillaries. In other instances, a fistula may be formed between two veins to form a veno-venous fistula. Generally, arterio-venous fistula formation requires surgical dissection of a target vein, and transecting and moving the vein for surgical anastomosis to the artery. It may therefore be useful to find less invasive and reliable devices and methods for aligning blood vessels and forming a fistula between the aligned blood vessels.

### SUMMARY

One challenging aspect of forming a fistula (endovascular treatment) between blood vessels, or other body vessels, is maneuvering a treatment portion of a catheter through tortuous vessels. Embodiments of the present disclosure are directed to systems, methods, and catheters for fistula formation that provide improved flexibility, as will be described in greater detail below.

In one embodiment, a catheter for use in a system for forming a fistula includes a catheter body, and a segmented backstop coupled to the catheter body and configured to receive a fistula-forming element. The segmented backstop includes at least a proximal segment and a distal segment, independently articulable relative to one another.

In another embodiment, a system for forming a fistula between two blood vessels includes a first catheter configured to be received in a first blood vessel and a second catheter configured to be received in a second blood vessel. The first catheter includes a first catheter body, and a fistula-forming element. The second catheter includes a second catheter body, and a segmented backstop coupled to the second catheter body and configured to receive the fistula-forming element. The segmented backstop includes at least a proximal segment and a distal segment, independently articulable relative to one another.

In yet another embodiment, a method for forming a fistula between two blood vessels includes advancing a first catheter into a first blood vessel, advancing a second catheter into a second blood vessel adjacent to the first blood vessel, and forming a fistula between the first blood vessel and the second blood vessel. The first catheter includes a first catheter body and a fistula-forming element. The second catheter includes a second catheter body and a segmented backstop coupled to the second catheter body, the segmented backstop including at least a proximal segment and a distal segment, independently articulable relative to one another. The segmented backstop is configured to articulate as the second catheter is advanced into the second blood vessel and receive the fistula-forming element of the first catheter. Forming a fistula between the first blood vessel and the second blood vessel is achieved by engaging the fistula forming element with the segmented backstop.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts end portions of a system of catheters for endovascular treatment of a blood vessel, according to one or more embodiments shown and described herein;

FIG. 2A schematically depicts a more detailed view of the backstop of the catheter of FIG. 1, according to one or more embodiments shown and described herein;

FIG. 2B schematically depicts flexure of the backstop of FIG. 2A, according to one or more embodiments shown and described herein;

FIG. 3 schematically depicts another embodiment of a backstop of a catheter that may be incorporated into the system of FIG. 1, according to one or more embodiments shown and described herein;

FIG. 4 schematically depicts another embodiment of a backstop of a catheter that may be incorporated into the system of FIG. 1, according to one or more embodiments shown and described herein

FIG. 5 schematically depicts another embodiment of a backstop of a catheter that may be incorporated into the system of FIG. 1, according to one or more embodiments shown and described herein;

FIG. 6 schematically depicts flexure of the backstop of FIG. 5, according to one or more embodiments shown and described herein;

FIG. 7 schematically depicts a method for treating a blood vessel, according to one or more embodiments shown and described herein;

FIG. 8 schematically depicts an end portion of a first catheter of the system of FIG. 1 being advanced through a first blood vessel and an end portion of a second catheter of the system of FIG. 1 being advanced through a second vessel, according to one or more embodiments shown and described herein;

FIG. 9 schematically depicts alignment of the first catheter and the second catheter of FIG. 8, according to one or more embodiments shown and described herein;

FIG. 10 schematically depicts treatment of the first blood vessel and the second blood vessel with the first catheter and the second catheter of FIG. 9, according to one or more embodiments shown and described herein; and

FIG. 11 schematically depicts a fistula formed between the first blood vessel and the second blood vessel, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to devices, systems, and methods for endovascular treatment of a blood vessel such as, but not limited to forming a fistula, wire crossing procedures, bypass procedures, etc. For example, a catheter may be placed in each of two adjacent blood vessels to form a fistula therebetween with the catheters. However, flexibility of the catheters, spacing of vessels, thickness of the vessel walls, and/or the tortuous anatomy of the vessels, may make it difficult to provide sufficient coaptation and/or alignment between vessels for fistula formation. Embodiments of the present disclosure provide improved flexibility of the catheters thereby providing improved maneuverability of the catheters through tortuous anatomy of vessels. For example, in some embodiments, a catheter for endovascular treatment of a blood vessel according to the present disclosure includes a segmented backstop for receiving a portion of another catheter that forms the fistula between two adjacent vessels, the segmented backstop may include a proximal segment and a distal segment independently articulable relative to one another. The segmented backstop allows for increased flexibility when maneuvering through vessels. These and additional features and benefits will be described in greater detail herein.

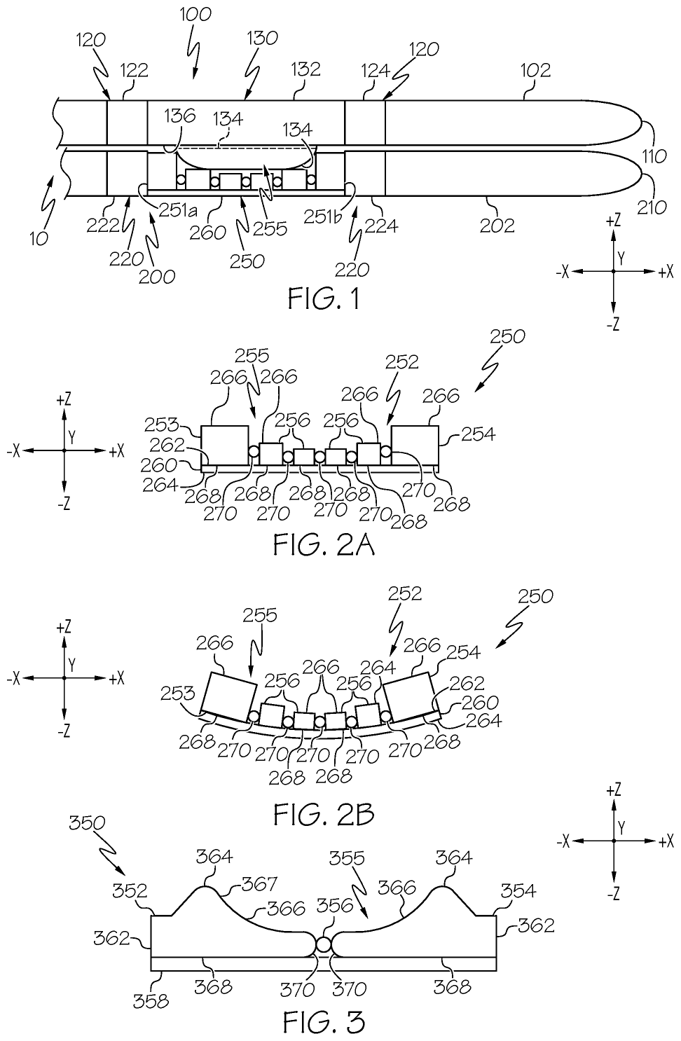

Referring now to FIG. 1, a system 10 for providing endovascular treatment of a blood vessel, such as fistula formation, is schematically depicted. The system 10 may include a first catheter 100 and a second catheter 200. It may be appreciated that aspects of the first catheter 100 and the second catheter 200 are substantially similar to one another. Accordingly, description of the first catheter 100 generally applies to the second catheter 200 unless otherwise noted or apparent. It is noted that the first catheter 100 and the second catheter 200 may be provided within a kit and/or separately from one another.

The first catheter 100 may generally include a first catheter body 102, a treatment portion 130, and one or more alignment elements 120. The first catheter 100 may be configured to be received in a first blood vessel 700 of a subject. It is noted that the first catheter 100 may include a greater or fewer number of components without departing from the scope of the present disclosure.

The first catheter body 102 may be sized to be advanced through a blood vessel and may include a distal tip 110 that may be shaped and/or sized to aid in advancement of the first catheter 100 through a blood vessel. For example, the distal tip 110 may be atraumatic for advancement through a blood vessel and may be any suitable shape, such as pointed, tapered, or the like. The first catheter body 102 may have any cross-sectional shape and any diameter suitable for intravascular use. The first catheter 100 may include or define one or more lumens or other passageways (not shown) extending at least partially along or through the first catheter body 102. For instance, the one or more lumens may extend at least partially longitudinally through the first catheter body 102. The first catheter body 102 may be formed of any material or combination of materials able to be traversed through a vasculature of a body. For example, the first catheter body 102 may include silicone, rubber, or the like.

As noted above, the first catheter 100 may have a treatment portion 130 for endovascular treatment of a blood vessel. In embodiments, the treatment portion 130 may have a housing 132 and a fistula-forming element 134. In embodiments, the fistula-forming element 134 may be an electrode, such as a leaf spring electrode, having an exposed ablation surface. The fistula-forming element 134 may be coupled to a power source (not shown), such as via a lead wire or other conductor attached thereto. When activated, current may be supplied to and/or carried from tissue and fluid via the ablation surface to facilitate ablation or vaporization of tissue to form a fistula.

In some embodiments, the fistula-forming element 134 may be a spring wire or leaf spring electrode, which may be movable between a retracted configuration (shown in phantom), in which the fistula-forming element 134 is retained within the first catheter 100 or positioned more closely to the housing 132, as depicted, and an expanded configuration, in which the fistula-forming element 134 projects beyond the outer surface 136 of the first catheter body 102 a distance greater than any projection from housing as compared to the retracted configuration. The fistula-forming element 134 may or may not be naturally biased to project from the housing 132. When the fistula-forming element 134 is naturally biased to project from the housing 132, a structure, such as a sleeve (not shown), may be used to hold or maintain the fistula-forming element 134 in a retracted configuration until deployment is desired. In some embodiments, the fistula-forming element 134 may be manually advanced or retracted such as via a lead wire which may be coupled to the fistula-forming element 134 which may be manually pulled or pushed to retract or advance the fistula-forming element 134. In some embodiments, the first catheter 100 may comprise one or more insulating materials (not shown) which may shield or otherwise protect the first catheter 100 and its components from heat generated by the fistula-forming element 134 during use. The insulating materials may be a coating layer on the outer surface of the fistula-forming element 134, or may surround the fistula-forming element 134. The insulating materials may be any material capable of thermally insulating the surrounding components such as, for example, metal.

As depicted, the fistula-forming element 134 may be arc shaped, though other shapes are contemplated and possible (e.g., rectangular, square, angular, etc.). The size and shape of the fistula-forming element 134 may be varied based on factors including tissue thickness and density, as well as desired fistula size, shape, and location. It is noted that the fistula-forming element 134 is not limited to an electrode as described above, but may include a different cutting/ablation device such as, but not limited to, any electrocautery mechanism, blades, lances, needles, cryogenic-cautery devices, ultrasonic-cautery devices, laser ablation devices, etc.

Still referring to FIG. 1 and as noted above, the first catheter 100 may include one or more alignment elements 120 configured to aid in alignment and/or coaptation of the first catheter 100 and the second catheter 200. For example, and not as a limitation, the one or more alignment elements may include a proximal magnetic portion 122 such as a proximal magnetic array arranged on or within the first catheter body 102 proximal to and adjacent the treatment portion 130. The proximal magnetic array may include a plurality of magnetic elements arranged in a longitudinal array along a length of the catheter. Similarly, the one or more alignment elements 120 may include a distal magnetic portion 124 such as a distal magnetic array arranged on or within the first catheter body 102 distal to and adjacent the treatment portion 130. The distal magnetic array may include a plurality of magnetic elements arranged in a longitudinal array along the length of the first catheter 100.

Each of the proximal and distal magnetic elements 122, 124 may have substantially the same dimensions (e.g., height, width, and depth) and substantially the same magnetic strength. In some embodiments, the proximal and distal magnetic elements 122, 124 may have a combination of different sized magnets, different shaped magnets, and/or magnets of differing magnetic strength. For example, the individual magnetic elements may have a substantially cubic shape, circular shape, oval shape, or any shape configured to fit within a targeted blood vessel.

In some embodiments, the number of the proximal and distal magnetic elements 122, 124 may be modified for optimization of magnetic strength for alignment or coaptation purposes. Each of the proximal magnetic portion 122 and the distal magnetic portion 124 may comprise any number of individual magnets including one or more magnets. Moreover, each catheter may comprise any number of individual magnets (e.g., one, two, three, four, five, six, seven, or eight or more, etc.). In embodiments, the proximal magnetic portion 122 and the distal magnetic portion 124 may have different lengths (e.g., different numbers of magnetic elements). For example, the proximal magnetic portion 122 may have a length that is longer than a length of the distal magnetic portion 124.

Each of the proximal magnetic portion 122 and the distal magnetic portion 124 may be disposed directly adjacent to the treatment portion 130. It is noted that in some embodiments, the proximal magnetic portion 122 and the distal magnetic portion 124 may not be positioned directly adjacent the treatment portion 130 but may be spaced from the treatment portion 130.

It is contemplated and possible that the one or more alignment elements 120 may include other alignment means, such as, for example, balloons, biasing rails, expandable cages, or the like. The other alignment means may be in addition, or alternative, to the proximal magnetic portion 122 and the distal magnetic portion 124. The alignment elements 120 may aid in alignment and/or coaptation between the first catheter and the second catheter.

Referring now to FIGS. 1 and 2A, the second catheter 200 may generally include a second catheter body 202, a segmented backstop 250, and one or more alignment elements 220 configured to aid in alignment and/or coaptation of the first catheter 100 and the second catheter 200. It is noted that a greater or fewer number of components may be included in the second catheter 200 without departing from the scope of the present disclosure.

The second catheter body 202 may include a distal tip 210 that may be shaped and/or sized to aid in advancement of the second catheter 200 through a blood vessel. For example, the distal tip 210 may be atraumatic for advancement through a blood vessel, and be any suitable shape, such as pointed, tapered, or the like. The second catheter body 202 may have any cross-sectional shape and any diameter suitable for intravascular use. The second catheter 200 may include or define one or more lumens or other passageways (not shown) extending at least partially along or through the second catheter body 202. For instance, the one or more lumens may extend at least partially longitudinally through the second catheter body 202. The second catheter body 202 may be formed of any material or combination of materials able to be traversed through a vasculature of a body. For example, the second catheter body 202 may include, silicone, rubber, or the like.

For example, and not as a limitation, the one or more alignment elements 220 may include a proximal magnetic element 222 such as a proximal magnetic array arranged on or within the second catheter body 202 proximal to and adjacent the segmented backstop 250. The proximal magnetic array may include a plurality of magnetic elements arranged in a longitudinal array along a length of the catheter. Similarly, the one or more alignment elements 220 may include a distal magnetic element 224 such as a distal magnetic array arranged on or within the second catheter body 202 distal to and adjacent the segmented backstop 250. The distal magnetic array may include a plurality of magnetic elements arranged in a longitudinal array along the length of the catheter. It is noted that the second catheter 200 may include a greater or fewer number of components without departing from the scope of the present disclosure.

Each of the proximal and distal magnetic elements 222, 224 may have substantially the same dimensions (e.g., height, width, and depth) and substantially the same magnetic strength. In some embodiments, the proximal and distal magnetic elements 222, 224 may have a combination of different sized magnets, different shaped magnets, and/or magnets of differing magnetic strength. For example, the individual magnetic elements may have a substantially cubic shape, circular shape, oval shape, or any shape configured to fit within a targeted blood vessel.

In some embodiments, the number of the plurality of proximal and distal magnetic elements 222, 224 may be modified for optimization of magnetic strength for alignment or coaptation purposes. Each of the proximal magnetic element 222 and the distal magnetic element 224 may comprise any number of individual magnetic elements including one or more magnetic elements. Moreover, each catheter may comprise any number of individual magnetic elements (e.g., one, two, three, four, five, six, seven, or eight or more, etc.). In embodiments, the proximal magnetic element 222 and the distal magnetic element 224 may have different lengths (e.g., different numbers of magnetic elements). For example, the proximal magnetic element 222 may have a length that is longer than a length of the distal magnetic element 224. The length of the proximal magnetic element 222 of the second catheter 200 may be substantially the same as the length of the proximal magnetic portion 122 of the first catheter 100. Similarly, the length of the distal magnetic element 224 of the second catheter 200 may be substantially the same as the length of the distal magnetic portion 124 of the first catheter 100.

Each of the proximal magnetic element 222 and the distal magnetic element 224 of the second catheter 200 may be disposed directly adjacent to the segmented backstop 250. It is noted that in some embodiments, the proximal magnetic element 222 and the distal magnetic element 224 of the second catheter 200 may not be positioned directly adjacent the segmented backstop 250 but may be spaced from the segmented backstop 250.

It is contemplated and possible that the one or more alignment elements 220 may include other alignment means, such as, for example, balloons, biasing rails, expandable cages, or the like. The other alignment means may be in addition, or alternatively, to the proximal magnetic element 222 and the distal magnetic element 224. The alignment elements 220 may aid be provided for alignment and/or coaptation between the first catheter 100 and the second catheter 200.

The segmented backstop 250 may be coupled to the second catheter body 202 at a first end 251a of the backstop 250 and at a second end 251b of the backstop 250. For example, the segmented backstop 250 may be adhered, welded, fastened, or the like to the second catheter body 202 and/or the alignment elements arranged on either side of the segmented backstop 250. The segmented backstop 250 may include a plurality of segments 252 which may be pivotable relative to one another to allow the segmented backstop 250 to flex and bend for traversal through tortuous anatomy. FIGS. 2A and 2B depict a more detailed view of the backstop 250. As depicted, the segmented backstop 250 may further include a substrate 260 and/or one or more spacers 270.

The plurality of segments 252 may include at least a proximal segment 253 and a distal segment 254. In embodiments, and as depicted, the plurality of segments 252 may further include one or more intermediate segments 256 positioned between the proximal segment 253 and the distal segment 254. The plurality of segments 252 may be independently articulable relative to one another. For example, each of the plurality of segments may be spaced apart from one another or may be otherwise shaped to allow articulation between each segment of the plurality of segments. For example, each of the proximal segment 253 and the distal segment 254 may independently articulate relative to one another. Moreover, any intermediate segments 256 may also be independently articulable relative to the proximal segment 253, the distal segment 254, and/or each other. The segmented backstop 250 may include any number of segments such as two segments (e.g., the proximal segment 253 and the distal segment 254. In embodiments include intermediate segments 256, there may be any number of intermediate segments 256, such as, for example, one, two, three, four, etc. In embodiments, the backstop 250 may not include intermediate segments 256.

The size and shape of the segmented backstop 250 may be varied based on factors including tissue thickness and density, as well as desired fistula size, shape, and location. For example, the proximal segment 253, the distal segment 254, and/or the intermediate segments 256 may be shaped to collectively define a recess 255. The proximal segment 253, the distal segment 254, and the intermediate segments 256 may be shaped such that the recess 255 may comprise a shape complementary to a shape of the fistula-forming element 134. For example, the recess 255 may comprise a saddle shape. Each of the proximal segment 253, the distal segment 254, and/or the intermediate segments 256 may be shaped to have rectangular cross-sections. However, in embodiments, the segments may include any shape, such as circular, triangular, pentagonal, or the like. The segments may include additional shapes, as will be described later with reference to FIGS. 3-6.

To aid in providing the recess 255, each of the proximal segment 253, the distal segment 254, and the intermediate segments 256 may have different heights in the +/−Z direction of the depicted coordinate axes. For example, the distal segment 254 and the proximal segment may have a substantially equal height, which the intermediate segments 256 have shorter heights. In embodiments including more than one intermediate segments 256, the intermediate segments 256 may include different heights among the intermediate segments. For example, the intermediate segments 256 that are centermost of the proximal segment 253 and the distal segment 254 may have a height that is less than a height of the intermediate segments 256 that are positioned between the centermost intermediate segments 256 and the proximal segment 253 or the distal segment 254.

Each of the proximal segment 253, the distal segment 254, and the intermediate segments 256 may be formed of, for example, ceramic. However, the plurality of segments 252 may be formed of any material, such as a metal, a polymer, a plastic, or the like. In some embodiments, each segment of the plurality of segments may be formed of the same or different materials.

Each of the proximal segment 253, the distal segment 254, and the intermediate segments 256 may be arranged such that lower surfaces 268 of each of the proximal segment 253, the distal segment 254, and the intermediate segments 256 are coplanar when arranged on a flat surface. In embodiments where the heights of the proximal segment 253, the distal segment 254 differ, upper surfaces 266 of the proximal segment 253, the distal segment 254, and the intermediate segments 256 may be noncoplanar such as to define the recess 255.

As noted above, the segmented backstop 250 may include the substrate 260. The substrate 260 may generally define a first surface 262, and a second surface 264 opposite the first surface 262. The first surface 262 may be coupled to the plurality of segments 252 of the segmented backstop 250 by, for example, welding, brazing, fasteners, adhesive, or the like. The substrate 260 may be formed of a flexible material such as, for example, a polymer, a plastic, a metal, or the like. In some embodiments, the substrate 260 may be formed from the second catheter body 202 of the second catheter 200. The first surface 262 of the substrate 260 may be coupled to lower surfaces 268 of each of the proximal segment 253, the distal segment 254, and/or the intermediate segments 256. In some embodiments, the substrate 260 may be coupled to the second catheter body 202 and/or the alignment elements 222, 224 via welding, brazing, fasteners, adhesive, or the like at a pair of ends flanking the first surface 262. In embodiments, the substrate 260 may be coupled to the catheter body 202 by a wire extending through the substrate 260 and the catheter body 202. The substrate 260 may be formed of an extendible material such that the substrate 260 may expand during flexion of the backstop 250.

In embodiments, the plurality of segments 252 may be mounted to the substrate 260 such that there is a space or void positioned between each segment of the plurality of segments. Accordingly, the flexibility of the substrate 260 and spacing between the plurality of segments 252 may aid in allowing the plurality of segments 252 to independently articulate which remaining coupled to one another.

In embodiments, the one or more spacers 270 may be disposed between each adjacent proximal segment 253, intermediate segments 256, and/or distal segment 254 and support independent articulation of each of the plurality of segments 252. In some embodiments, the spacers 270 may maintain a spacing between each adjacent proximal segment 253, intermediate segments 256, and/or distal segment 254, and provide pivot locations about which the plurality of segments 252 may pivot. Accordingly, in embodiments, the one or more spacers 270 may be rounded to support articulation. The spacers 270 may be coupled to the intermediate segments 256, the proximal segment 253, and/or the distal segment 254. In embodiments, the spacers 270 may be coupled to the substrate 260 and be positioned to be in contact with each of the segments 252 to support articulation of each of the segments 252. In other embodiments, the spacers 270 may include a wire that extends through each of the spacers 270 and the segments 252 to couple each of the spacers 270 to the segments 252. It is noted that the second catheter 200 may include additional or alternative spacing members, aside from spacers 270. For example, the spacers 270 may be one or more bearings, hinges, magnets, or the like. The spacers 270 may be formed of a compressible material such that the spacers 270 may compress during flexion or compression of the backstop 250. In some embodiments, the segments 252 may be spaced apart without the use of spacers 270.

Referring specifically to FIG. 2B, the segmented backstop 250 may flex and contract due to the independently articulable proximal segment 253, distal segment 254, and intermediate segments 256 to conform to the geometry of a blood vessel. Referring to FIG. 2B, the segmented backstop 250 may contract such that upper surfaces 266 of the segments move inward toward a center of the backstop 250, and such that the lower surfaces 268 are no longer coplanar. The backstop 250 may similarly contract in an opposite direction, such that the upper surfaces 266 move outward away from the center of the backstop 250, and such that the lower surfaces 268 move inward toward each other. While FIG. 2B depicts the backstop 250 flexing in a single direction, the segments may independently articulate such that each adjacent segment articulates away from the adjacent segments in a different direction (e.g., in the +/−Y direction, +/−Z direction, and/or +/−X direction). This may occur during navigation of tortuous anatomy, where each segment may articulate separately to traverse a vessel.

Referring now to FIG. 3, another embodiment of a segmented backstop 350 is depicted. As will be appreciated by one of skill in the art, the segmented backstop 350 may be incorporated into the second catheter of FIG. 1 in place of the backstop 250. The backstop 350 may include a spacer 356, a substrate 358, a proximal segment 352, and a distal segment 354, though a greater or fewer number of components are contemplated. The backstop 350 is substantially similar to the backstop 250 as described above, and discussion of similar elements will not be discussed again for brevity.

As depicted, the backstop 350 includes the proximal segment 352 and the distal segment 354, however, the backstop 350 may include additional segments without departing from the scope of the present disclosure. The proximal segment 352 and the distal segment 354 may be shaped to define a recess 355. Each of the proximal segment 352 and the distal segment 354 may include a heel portion 362, a peak portion 364, a curved portion 366, a bottom surface 368, and a toe portion 370. The curved portion 366 may extend from the toe portion 370 to the peak portion 364. The curved portion 366 may include a curved upper surface 367 that defines the recess 355 of the backstop 350. The peak portion 364 may have a distance from the bottom surface 368 that is greater than a distance of the curved portion 366, the heel portion 362, or the toe portion 370 from the bottom surface 368.

The toe portion 370 of each of the proximal segment 352 and the distal segment 354 may contact and be coupled to the spacer 356 positioned between the proximal segment 352 and the distal segment 354. The proximal segment 352 and the distal segment 354 may be symmetrical to one another about the spacer 356. The substrate 358 may extend across and be coupled to the bottom surface 368 of each of the proximal segment 352 and the distal segment 354, thereby coupling the proximal segment 352 and the distal segment 354 together.

While the backstop 350 is depicted as including the spacer 356 and the substrate 358, the backstop 350 may include more or less components without departing from the scope of the disclosure. For example, the backstop 350 may not include the spacer 356 and/or the substrate 358. In such embodiments, the proximal segment 352 and the distal segment 354 may be connected by, for example, a hinge pin extending through the segments 352, 354.

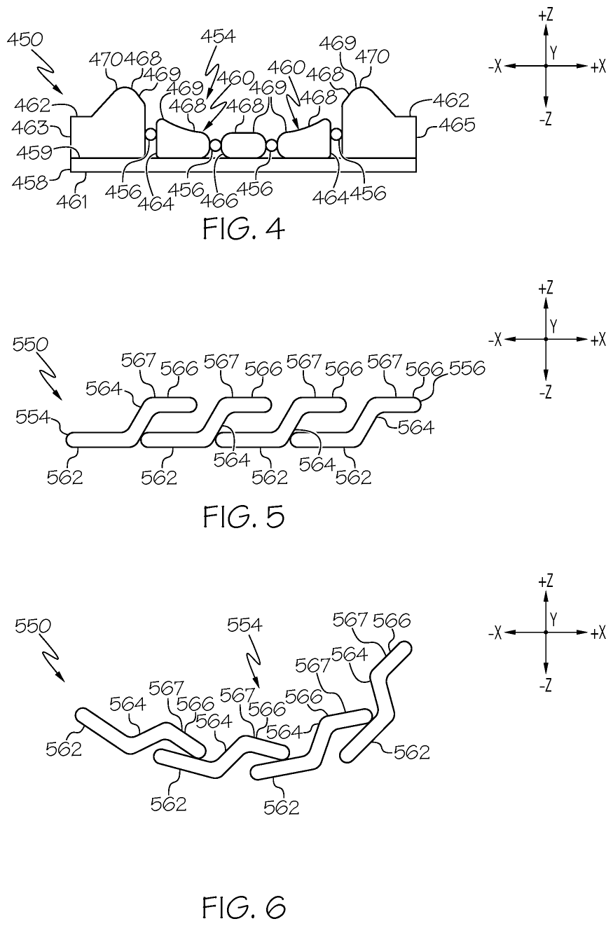

Referring now to FIG. 4, another alternative backstop 450 is depicted. The backstop 450 is substantially similar to the backstop 250 and the backstop 350 as described above, and discussion of similar elements will not be discussed again for brevity. As will be appreciated by one of skill in the art, the segmented backstop 450 may be incorporated into the second catheter of FIG. 1 in place of the backstop 250 or 350. As depicted, the backstop 450 includes a pair of outermost segments 462, an innermost segment 466, and a pair of intermediate segments 464 positioned between the outermost segments 462 and the innermost segment 466, a plurality of spacers 456, and a substrate 458. The plurality of spacers 456 may be positioned between each of the segments such that each of the segments are spaced apart. The substrate 458 may define a first surface 459 and a second surface 461. The segments may be coupled to the first surface 459 of the substrate 458. The pair of outermost segments 462 include a proximal segment 463 and a distal segment 465. However, the backstop 450 may include more or less segments without departing from the scope of the present disclosure. Additional segments allows for increased flexibility of the backstop 450 as each space between adjacent segments acts as a joint for the adjacent segments to move and/or rotate about.

The segments of the backstop 450 may be similarly shaped to the segments of the backstop 350. Specifically, the segments may include a curved portion 468 that extends from one of the outermost segments 462 to the other of the outermost segments 462. The curved portion 468 may be formed in each of the segments. The curved portion 468 may include a curved surface 469 that across each of the segments to collectively define a recess 454 of the backstop 450. The segments may further include peak portions 470 on opposing sides of the curved portion 468. The curved portion 468 may extend from one of the peak portions 470 to the other of the peak portions 470. The peak portions 470 may be formed in the outermost segments 462.

Referring now to FIGS. 5 and 6, another backstop 550 is depicted. As will be appreciated by one of skill in the art, the segmented backstop 550 may be incorporated into the second catheter of FIG. 1 in place of the backstop 250, 350, or 450. The backstop 550 includes a plurality of segments including a proximal segment 554 and a distal segment 556. The backstop 550 may further include additional segments disposed between the proximal segment 554 and the distal segment 556. The proximal segment 554 and the distal segment 556, as well as any intervening segments, may be independently articulable relative to one another. Each of the segments of the plurality of segments may be substantially identical to one another. For example, each of the plurality of segments may be Z-shaped and include a first extending portion 562, a second extending portion 566, and an angled portion 564 extending between and connecting the first extending portion and the second extending portion. The angled portion 564 may extend from the first extending portion 562 to the second extending portion 566 at an oblique angle. The first extending portion 562 and the second extending portion 566 may extend from the angled portion 564 such that the first extending portion 562 is parallel to second extending portion 562. A length of the first extending portion 562 (in the +/−Y direction of the depicted coordinate axes) may be greater than a length of the second extending portion 566 or vice versa. In some embodiments, it is contemplated that the first extending portion 562 and the second extending portion 566 may be the same length. Referring to FIG. 5, the proximal segment 554 and the distal segment 556 as well as any intervening segments may define a flat surface 567 extending across the second extending portions 566 when the plurality of segments are arranged linearly, as depicted. Referring to FIG. 6, the flat surface 567 may be configured to conform to a shape of the fistula-forming element 134 when acted upon by a bending force. The bending force may be from the fistula-forming element 134 being pressed into the backstop 550 and/or traversal of tortuous anatomy of a vessel. The second extending portion 566 of the proximal segment 554 may overlap the first extending portion 562 of the distal segment 556, or an adjacent segment in the +/−Z direction of the depicted coordinate axes.

As noted above, the plurality of segments may articulate relative to one another. For example, the plurality of segments may be coupled to one another via a hinge, a conformable sleeve containing the segments, a flexible substrate on which the segments are formed, or the like. In embodiments, the backstop 550 may include a wire that extends through each of the segments to couple the segments together.

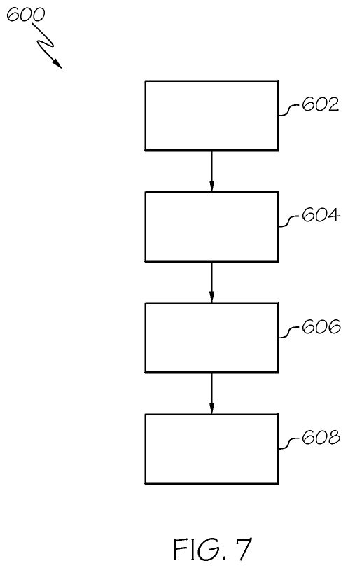

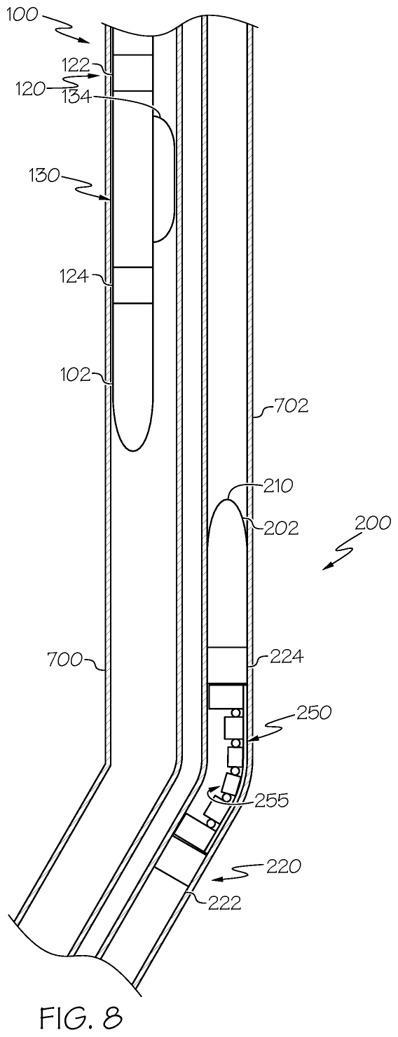

Referring now to FIG. 7, a flow chart depicting a method 600 of forming a fistula is generally depicted. It is noted that the method 600 may include a greater or fewer number of steps, taken in any order, without departing from the scope of the present disclosure. It is noted that the method 600 illustrated in FIG. 7 may be best understood when reviewed in conjunction with FIGS. 8-11 which generally illustrate alignment and treatment of a blood vessel using the first and second catheters 100, 200 as described herein. It is noted that while the segmented backstop 250 of FIGS. 1-2B is generally depicted, the segmented backstop could instead be any embodiment of the segmented backstop described herein.

Still referring to FIG. 7, at block 602 the method 600 includes advancing the first catheter 100 through a first blood vessel 700 as depicted in FIG. 8. For example, a user may advance the first catheter 100 through the first blood vessel 700 to a first treatment location so that the treatment portion 130 is advanced to the first treatment location.

Referring again to FIG. 7, at block 604 the method 600 further includes advancing the second catheter 200 through a second blood vessel 702. For example, and as depicted in FIG. 8, the user may advance the second catheter 200 through the second blood vessel 702 to a second treatment location in the second vessel. It is noted that the first blood vessel 700 and the second blood vessel 702 may be adjacent vessels such as a vein and an artery, though vein to vein and artery to artery treatments are contemplated and possible. While advancing the second catheter 200 through the second blood vessel 702, pressure from the second blood vessel 702 onto the backstop 250 may cause or allow each of the segments of the backstop 250 to articulate, thereby shaping the backstop 250 to conform to the shape of the second blood vessel 702, thereby improving the ability of the second catheter 200 to be traversed through the tortuous anatomy of the second blood vessel 702.

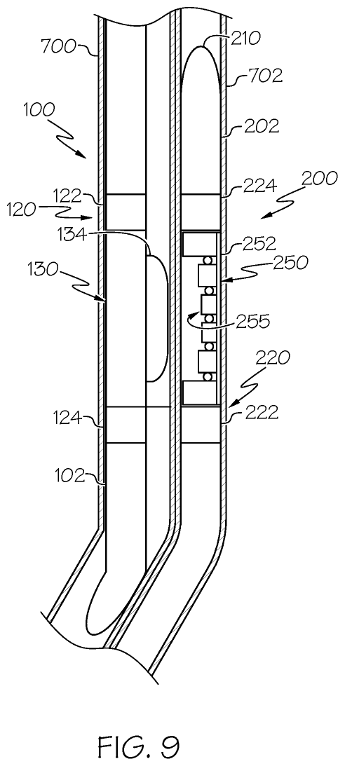

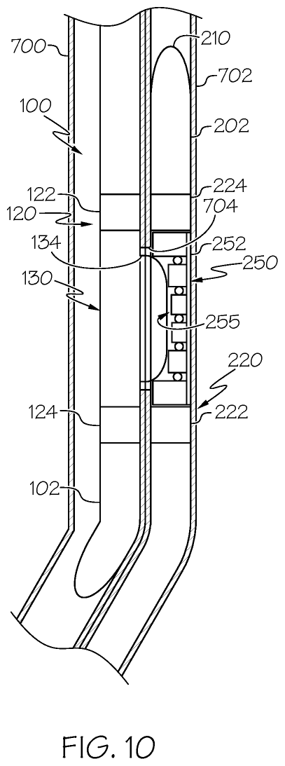

Referring again to FIG. 7, at block 606 the method 600 includes aligning the treatment portion 130 of the first catheter 100 with the segmented backstop 250 of the second catheter 200. For example, as depicted in FIGS. 9 and 10, once the treatment portion 130 and the backstop 250 of the first catheter 100 and the second catheter 200, respectively, are aligned (e.g., both longitudinally and rotationally) the first and second catheters 100, 200 may become attracted to one another via attraction between the proximal magnetic portion 122 of the first catheter 100 and the proximal magnetic element 222 of the second catheter 200 and attraction between the distal magnetic portion 124 of the first catheter 100 and the distal magnetic element 224 of the second catheter 200. The attraction between the first and second catheters 100, 200 may cause the first catheter 100 and the second catheter 200 to snap together, thereby pulling the first blood vessel 700 and the second blood vessel 702 into contact with one another, as illustrated in FIG. 10. Accordingly, once aligned, the first catheter 100 and the second catheter 200 may coapt together and sandwich tissue for the first vessel and the second vessel therebetween. The snapping effect caused by the magnetic attraction between the first catheter 100 and the second catheter 200 may cause tactile feedback which may be felt by a user and provide an indication of proper longitudinal alignment between the first catheter 100 and the second catheter 200. In some embodiments, the method 600 may further include confirming alignment of the first catheter 100 and the second catheter 200 via fluoroscopy or other imaging techniques.

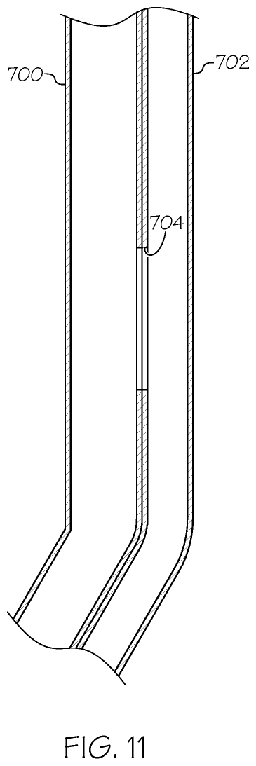

Referring again to FIG. 7, at block 608 the method 600 may further include, after alignment between the treatment portion 130 and the backstop 250 of the first catheter 100 and the second catheter 200, respectively, performing a vascular treatment procedure to modify the first blood vessel 700 and/or the second blood vessel 702 at the first treatment location and/or the second treatment location. For example, the fistula-forming element 134 of the treatment portion 130 may be activated to ablate tissue of the first blood vessel 700 and the second blood vessel 702 to form a fistula 704 between the first blood vessel 700 and the second blood vessel 702, as generally depicted in FIGS. 10 and 11. As the fistula-forming element 134 advances through the tissue of the first blood vessel 700 and the second blood vessel 702,, the fistula forming element of the first catheter 100 engages and is received by the segmented backstop 250 of the second catheter 200 by extending into the recess 255 of the segmented backstop 250. Upon formation of the fistula 704, the first catheter 100 and the second catheter 200 may be withdrawn from the respective first blood vessel 700 and the second blood vessel 702.

### Embodiments may be Further Described With Reference to the Following Numbered Clauses

1. A catheter for use in a system for forming a fistula, the catheter comprising: a catheter body; and a segmented backstop coupled to the catheter body and configured to receive a fistula-forming element, wherein the segmented backstop comprises at least a proximal segment and a distal segment, independently articulable relative to one another.
2. The catheter of the preceding clause, wherein the proximal segment and the distal segment together define a recess comprising a shape complimentary to a shape of the fistula-forming element.
3. The catheter of any of the preceding clauses, wherein the proximal segment and the distal segment together define a recess comprising a saddle shape.
4. The catheter of any of the preceding clauses, wherein the segmented backstop comprises one or more additional segments positioned between the proximal segment and the distal segment.
5. The catheter of any of the preceding clauses, wherein each additional segment is independently articulable relative to the proximal segment and the distal segment.
6. The catheter of any of the preceding clauses, wherein the catheter further comprises a substrate that defines a first surface and a second surface; the first surface is coupled to the segmented backstop; and the second surface is coupled to the catheter body.
7. The catheter of any of the preceding clauses, wherein the catheter further comprises one or more spacers disposed between the proximal segment and the distal segment.
8. The catheter of any of the preceding clauses, wherein the proximal segment and the distal segment together form a substantially flat surface configured to conform to a shape of the fistula-forming element when acted upon by a bending force.
9. The catheter of any of the preceding clauses, wherein a portion of the proximal segment overlaps a portion of the distal segment to form the substantially flat surface.
10. A system for forming a fistula between two blood vessels, the system comprising: a first catheter configured to be received in a first blood vessel, the first catheter comprising: a first catheter body; and a fistula-forming element; a second catheter configured to be received in a second blood vessel adjacent to the first blood vessel, the second catheter comprising: a second catheter body; and a segmented backstop coupled to the second catheter body and configured to receive the fistula-forming element, the segmented backstop comprises at least a proximal segment and a distal segment, independently articulable relative to one another.
11. The system of the preceding clause, wherein the proximal segment and the distal segment together define a recess comprising a shape complimentary to a shape of the fistula-forming element.
12. The system of any of the preceding clauses, wherein the proximal segment and the distal segment together define a recess comprising a saddle shape.
13. The system of any of the preceding clauses, wherein the segmented backstop comprises one or more additional segments positioned between the proximal segment and the distal segment, wherein each additional segment is independently articulable relative to the proximal segment and the distal segment.
14. The system of any of the preceding clauses, wherein the second catheter further comprises a substrate that defines a first surface and a second surface; the first surface is coupled to the segmented backstop; and the second surface is coupled to the second catheter body.
15. The system of any of the preceding clauses, wherein the second catheter further comprises one or more spacers disposed between the proximal segment and the distal segment.
16. A method for forming a fistula between two blood vessels, the method comprising: advancing a first catheter into a first blood vessel, the first catheter comprising: a first catheter body; and a fistula-forming element; advancing a second catheter into a second blood vessel adjacent to the first blood vessel, the second catheter comprising: a second catheter body; and a segmented backstop coupled to the second catheter body, the segmented backstop comprising at least a proximal segment and a distal segment, independently articulable relative to one another, the segmented backstop is configured to articulate as the second catheter is advanced into the second blood vessel and receive the fistula-forming element of the first catheter; forming a fistula between the first blood vessel and the second blood vessel by engaging the fistula-forming element with the segmented backstop.
17. The method of any of the preceding clauses, wherein the proximal segment and the distal segment together define a recess comprising a shape complimentary to a shape of the fistula-forming element.
18. The method of any of the preceding clauses, wherein the segmented backstop comprises one or more additional segments positioned between the proximal segment and the distal segment, wherein each additional segment is independently articulable relative to the proximal segment and the distal segment.
19. The method of any of the preceding clauses, wherein the second catheter further comprises one or more spacers disposed between the proximal segment and the distal segment.
20. The method of any of the preceding clauses, wherein the proximal segment and the distal segment together form a substantially flat surface configured to conform to a shape of the fistula-forming element when acted upon by a bending force.
21. The method of any of the preceding clauses, wherein a portion of the proximal segment overlaps a portion of the distal segment to form the substantially flat surface.

It should now be understood that embodiments of the present disclosure are directed to devices, systems, and methods for forming a fistula between two blood vessels. In some embodiments, the devices and methods may be used to form a fistula between two blood vessels. More particularly, a catheter may be placed in each of two adjacent blood vessels to form a fistula therebetween with the catheters. For example, in some embodiments, a catheter for endovascular treatment of a blood vessel includes a backstop including a plurality of segmented shaping members that independently articulate relative to one another. These and additional features and benefits will be described in greater detail herein.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A catheter for use in a system for forming a fistula, the catheter comprising:
a catheter body; and
a segmented backstop coupled to the catheter body and configured to receive a fistula-forming element, wherein the segmented backstop comprises at least a proximal segment and a distal segment, independently articulable relative to one another.

2. The catheter of claim 1, wherein the proximal segment and the distal segment together define a recess comprising a shape complimentary to a shape of the fistula-forming element.

3. The catheter of claim 1, wherein the proximal segment and the distal segment together define a recess comprising a saddle shape.

4. The catheter of claim 1, wherein the segmented backstop comprises one or more additional segments positioned between the proximal segment and the distal segment.

5. The catheter of claim 4, wherein each additional segment is independently articulable relative to the proximal segment and the distal segment.

6. The catheter of claim 1, wherein the catheter further comprises a substrate that defines a first surface and a second surface; the first surface is coupled to the segmented backstop; and the second surface is coupled to the catheter body.

7. The catheter of claim 1, wherein the catheter further comprises one or more spacers disposed between the proximal segment and the distal segment.

8. The catheter of claim 1, wherein the proximal segment and the distal segment together form a substantially flat surface configured to conform to a shape of the fistula-forming element when acted upon by a bending force.

9. The catheter of claim 8, wherein a portion of the proximal segment overlaps a portion of the distal segment to form the substantially flat surface.

10. A system for forming a fistula between two blood vessels, the system comprising:
a first catheter configured to be received in a first blood vessel, the first catheter comprising:
a first catheter body; and
a fistula-forming element;
a second catheter configured to be received in a second blood vessel adjacent to the first blood vessel, the second catheter comprising:
a second catheter body; and
a segmented backstop coupled to the second catheter body and configured to receive the fistula-forming element, the segmented backstop comprises at least a proximal segment and a distal segment, independently articulable relative to one another.

11. The system of claim 10, wherein the proximal segment and the distal segment together define a recess comprising a shape complimentary to a shape of the fistula-forming element.

12. The system of claim 10, wherein the proximal segment and the distal segment together define a recess comprising a saddle shape.

13. The system of claim 10, wherein the segmented backstop comprises one or more additional segments positioned between the proximal segment and the distal segment, wherein each additional segment is independently articulable relative to the proximal segment and the distal segment.

14. The system of claim 10, wherein the second catheter further comprises a substrate that defines a first surface and a second surface; the first surface is coupled to the segmented backstop;
and the second surface is coupled to the second catheter body.

15. The system of claim 10, wherein the second catheter further comprises one or more spacers disposed between the proximal segment and the distal segment.

16. A method for forming a fistula between two blood vessels, the method comprising:
advancing a first catheter into a first blood vessel, the first catheter comprising:
a first catheter body; and
a fistula-forming element;
advancing a second catheter into a second blood vessel adjacent to the first blood vessel, the second catheter comprising:
a second catheter body; and
a segmented backstop coupled to the second catheter body, the segmented backstop comprising at least a proximal segment and a distal segment, independently articulable relative to one another, the segmented backstop is configured to articulate as the second catheter is advanced into the second blood vessel and receive the fistula-forming element of the first catheter;
forming a fistula between the first blood vessel and the second blood vessel by engaging the fistula-forming element with the segmented backstop.

17. The method of claim 16, wherein the proximal segment and the distal segment together define a recess comprising a shape complimentary to a shape of the fistula-forming element.

18. The method of claim 16, wherein the segmented backstop comprises one or more additional segments positioned between the proximal segment and the distal segment, wherein each additional segment is independently articulable relative to the proximal segment and the distal segment.

19. The method of claim 16, wherein the second catheter further comprises one or more spacers disposed between the proximal segment and the distal segment.

20. The method of claim 16, wherein the proximal segment and the distal segment together form a substantially flat surface configured to conform to a shape of the fistula-forming element when acted upon by a bending force.
